(19) United States
(12) Patent Application Publication
(10) Pub. No.: US 2006/0084939 A1
(43) Pub. Date: Apr. 20, 2006

(54) Articulation segment for a catheter

(76) Inventor: David J. Lentz, La Jolla, CA (US)

Correspondence Address: Att: NEIL K. NYDEGGER;NYDEGGER & ASSOCIATES, 348 Olive Street, San Diego, CA, 92103, US

(21) Appl. No.: 10/969,088

(22) Filed: Oct. 20, 2004

(51) Int. Cl. A61M 25/00

(52) U.S. Cl. 604/526; 264/138

(57) ABSTRACT

An articulating segment for a catheter includes a tube shaped member that is formed with a helical cut around the tube's axis. The cut extends through the member, between its outer and inner surfaces, and defines a pitch angle with the axis that can be varied according to the desired flexibility of the segment. A flexible coating is positioned on the outer surface of the tube to cover the helical cut and to provide a fluid-tight condition during articulation of the segment.

### FIELD OF THE INVENTION

The present invention pertains generally to catheters. More particularly, the present invention pertains to articulating segments that can be used to conform a catheter to the tortuous paths and configurations that are operationally necessary for the catheter to be positioned in, or to pass through the vasculature of a patient. The present invention is particularly, but not exclusively, useful as a fluid-tight articulating segment that can effectively serve as a cryo-chamber in the operation of a cryo-catheter.

### BACKGROUND OF THE INVENTION

In general, a catheter is any hollow, flexible tube that can be inserted into a body cavity, duct or vessel for any of a variety of purposes. In each case, to be effective, the catheter must be somehow controllable so that it can be properly positioned in the body. Additionally, a catheter must have the structural capability of performing its intended purpose once it has been properly positioned. Of the many different types of medical catheters that are presently being used, the so-called cryo-catheters are of particular interest for the present invention. As is well known, these catheters are used primarily for the purpose of cryo-ablating tissue in the vasculature of a patient.

Unlike other types of catheters, a cryo-catheter is unique in that it has a cooling segment. Preferably, the cooling segment of a cryo-catheter is capable of being cooled to temperatures as low as approximately eighty-five Kelvin. With this requirement in mind, several competing structural characteristics for the cooling segment of a cryo-catheter become of particular importance. For one, the cooling segment of a cryo-catheter needs to be made of a thermally conductive material. Such materials without modification, however, do not typically have the flexibility that is required for maneuvering a catheter through the vasculature of a patient. Thus, if a thermally conductive material such as stainless steel is to be used, it needs to be somehow structurally modified to achieve the required flexibility. This however, in turn, leads to a consideration of other requirements such as fluid confinement in the cooling segment, and resistance to increases in fluid pressure.

Whatever modifications may be required to construct an effective cryo-catheter, it is clear the resulting structure must be sufficiently strong to confine a pressurized fluid refrigerant in the cooling segment. With this in mind, appreciate that a cryo-catheter is essentially nothing more than a tube. Thus, to accomplish certain of the requirements mentioned above, it must have good “hoop strength” for confining the pressurized fluid. Further, because pressurized fluids are involved, the structure of the cooling segment must also be fluid-tight to prevent any leakage of the fluid refrigerant. At the same time, the cryo-catheter must remain sufficiently flexible so it can be maneuvered while being advanced through the vasculature of a patient. Finally, it must also be sufficiently strong to resist kinking.

In light of the above, it is an object of the present invention to provide an articulating segment for a cryo-catheter that is made with a thermally conductive material which is structurally modified to provide the required flexibility for use in an invasive catheter. Another object of the present invention is to provide an articulating segment for a cryo-catheter which is thermally conductive, is flexible and is sufficiently strong to contain a pressurized refrigerant fluid. Still another object of the present invention is to provide an articulating segment for a cryo-catheter which is relatively simple to manufacture, is easy to use and is comparatively cost effective.

### SUMMARY OF THE INVENTION

In accordance with the present invention, an articulating segment for use in the cooling chamber of a cryo-catheter includes a tube shaped member that has an outer surface and an inner surface. The tube shaped member defines an axis and it is formed with a helical cut that goes around the axis. Specifically, the cut extends through the wall of the tube shaped member between its outer surface and its inner surface. In addition to this tube shaped member, the articulating segment includes a flexible coating that is positioned on the outer surface of the tube shaped member. This flexible coating covers the helical cut and provides a fluid-tight condition for the lumen that is created in the articulating segment of the tube shaped member.

Structurally, the helical cut in the articulating segment defines a pitch angle (α) that is measured between the cut and the axis of the tube shaped member. Preferably, this pitch angle (α) is in a range between forty-five and ninety degrees. Within this range, the pitch angle (α) can be varied during manufacture to achieve a predetermined flexibility for the segment. More specifically, an increase in the pitch angle (α) will provide increased flexibility for the segment. As a practical matter, this flexibility can be increased to a point where the predetermined flexibility for the articulating segment allows the tube shaped member to be bent with a radius of curvature of approximately fifteen mm.

In line with the description given above, the articulating segment can be thought of as being formed by a flat, narrow, ribbon-like band that is wound into a spiral. Importantly, for enhanced strength, this band has a substantially rectangular-shaped cross-section that is bounded by the upper and lower surfaces, and by opposed first and second edges. In this structure, the helical cut is formed as a gap between the first and second edges of the band. For disclosure purposes, this gap has a depth between the outer and inner surfaces that is in a range of 0.1 mm to 0.2 mm, and it has a width substantially perpendicular to the depth that is formed in a range of 10 microns and 100 microns. As a practical matter, however, unless the articulating segment is being bent, the edges of the band will generally be in contact with each other and, consequently, there will be no effective gap width.

Preferably, the tube shaped member is made of stainless steel or Nitinol and the flexible coating is made of nylon or of a polymer material.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of the distal extension of a cryo-catheter that includes an articulating segment in accordance with the present invention, wherein portions are removed for clarity; and

FIG. 2 is an enlarged cross-sectional view of the articulating segment of the present invention as seen along the line 2-2 in FIG. 1.

### DESCRIPTION OF THE PREFERRED EMBODIMENTS

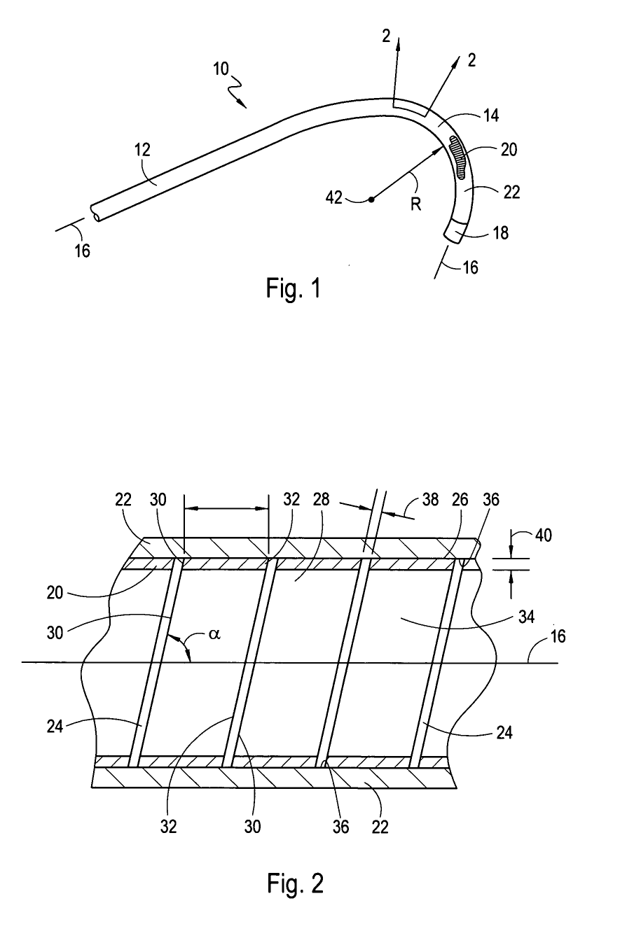

Referring initially to FIG. 1, the distal extension of a cryo-catheter is shown and generally designated 10. In this extension, the cryo-catheter 10 includes a shaft 12 and it has a distal segment 14. As indicated, the shaft 12 of catheter 10 generally defines an axis 16 that extends along the length of the catheter 10. For the specific case wherein the catheter 10 is a cryo-catheter, a cryo-tip 18 will be located at the extreme distal end of the catheter 10. This cryo-tip 18 will then establish a cooling chamber for the cryo-catheter 10 that extends proximally from the cryo-tip 18 back through a predetermined distance along the shaft 12. Insofar as the present invention is concerned, it is to be appreciated that the description of the catheter 10 as being a cryo-catheter is merely exemplary. Specifically, the present invention pertains to other type catheters as well.

Still referring to FIG. 1 it will be seen that, structurally, the shaft 12 and the distal segment 14 of cryo-catheter 10 include a tube shaped member 20 which is covered by a flexible coating 22. Preferably, the tube shaped member 20 is a so-called hypotube that is made of a highly thermally conductive material such as stainless steel or Nitinol. On the other hand, the flexible coating 22 is preferably made from a material such as Pebax or nylon. Although it is preferred that the flexible coating 22 be somewhat thermally conductive, it is perhaps more important that the flexible coating 22 have the lubricity and bio-compatibility properties which are required for medical catheters. Further, the flexible coating 22 should be made of a material that can be securely positioned on the tube shaped member 20. A more detailed structural description of the distal segment 14 is possible with reference to FIG. 2.

With reference to FIG. 2 it can be appreciated that the tube shaped member 20 is formed with a gap 24 which is helically shaped, and which is centered on the axis 16. Specifically, the gap 24 is created by a helical cut that extends between the outer surface 26 and the inner surface 28 of the tube shaped member 20. From a different aspect, the creation of gap 24, in turn, creates a substantially flat, narrow, ribbon-like band which is configured into a spiral to form the tube shaped member 20. Importantly, this band has a substantially rectangular cross-section that is bounded by the outer surface 26, the inner surface 28 and opposed edges 30 and 32.

Several operational benefits result from the rectangular shaped cross-section of the tube shaped member 20 in distal segment 14. For one, this configuration effectively maximizes the amount of material that is available to resist “hoop stress” in the distal segment 14. Thus, the tube shaped member 20 is able to contain higher fluid pressures inside the lumen 34 of tube shaped member 20 than might otherwise be possible. Further, the surface area 36 of flexible coating 22 in gap 24 that may be directly exposed to fluid pressures in lumen 34 is minimized.

As is also to be appreciated with reference to FIG. 2, the gap 24 can be geometrically varied during manufacture to obtain a predetermined flexibility for distal segment 14. Along with considerations of other dimensions of the tube shaped member 20 (e.g. its diameter), a pitch angle (α) can be established for the helical cut of gap 24 that will directly affect flexibility. Specifically, for a tube shaped member 20 having a given diameter, flexibility of the distal segment 14 can be increased or decreased by, respectively, using a larger or smaller pitch angle (α). Preferably, the pitch angle (α) will be selected in a range between approximately forty-five and ninety degrees.

Still referring to FIG. 2 it will be seen that the gap 24 is characterized as having a width 38 and a depth 40. In particular, the width 38 of the gap 24 will be determined by the amount of material that is removed during the manufacture of the distal segment 14. On the other hand, the depth 40 of gap 24 will depend on the thickness of the tube shaped member 20 that is selected for use in the manufacture of the distal segment 14. As intended for the present invention, the helical cut for gap 24 will be made by a laser beam using well known technology. Accordingly, the width 38 of gap 24 will preferably be manufactured to be in a range between approximately 10 microns and approximately 100 microns. The depth 40 of gap 24, however, will generally be in a range between approximately 0.1 mm and 0.2 mm. In any event, it is envisioned that the cross-section will be rectangular.

As disclosed above, a flexible coating 22 is positioned on the outer surface 26 of the tube shaped member 20 to cover the outer surface 26 and the gap 24. This then effectively provides a fluid-tight condition for the lumen 34. Additionally, as indicated above, the flexible coating 22 provides a degree of lubricity that will assist in the advancement of the catheter 10 into the vasculature of a patient.

In operation, the distal segment 14 of catheter 10 needs to be articulated for several reasons. These reasons include, steerability for the catheter 10 as it is being advanced to position the distal segment 14 at a site in the vasculature of a patient. Also, if the catheter 10 is a cryo-catheter, the distal segment 14 must be able to confine pressurized fluid refrigerants and be configurable to conform with tissue that is to be cryo-ablated. As envisioned for the present invention, these objectives are met by the ability of the structure for distal segment 14 to be bent on a curve of radius “R” as shown in FIG. 1. For the purposes of the present invention, the radius of curvature “R”, as measured from a center of curvature at point 42, may be as short as fifteen mm.

While the particular Articulation Segment for a Catheter as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. An articulating segment for a catheter which comprises:
a tube shaped member having an outer surface and an inner surface and defining an axis, wherein the member is formed with a helical cut around the axis with the cut extending through the member between the outer surface and the inner surface; and
a flexible coating positioned on the outer surface of the tube shaped member for covering the helical cut during articulation of the segment.

2. A segment as recited in claim 1 wherein the helical cut defines a pitch angle (α) between the cut and the axis.

3. A segment as recited in claim 2 wherein the pitch angle (α) between the cut and the axis is in a range between forty-five and ninety degrees.

4. A segment as recited in claim 1 wherein the inner surface of the tube shaped member defines a lumen for the segment and the flexible coating establishes a fluid-tight condition for the lumen.

5. A segment as recited in claim 1 wherein the helical cut has a depth between the outer surface and the inner surface and a width substantially perpendicular to the depth with the depth being in a range of 0.1 mm to 0.2 mm and the width being in a range of 10 microns and 100 microns.

6. A segment as recited in claim 1 wherein the tube shaped member is made of stainless steel.

7. A segment as recited in claim 1 wherein the tube shaped member is made of Nitinol.

8. A segment as recited in claim 1 wherein the flexible coating is made of a polymer material.

9. A segment as recited in claim 1 wherein the flexible coating is made of nylon.

10. An articulating segment for a catheter which comprises:
a substantially flat, narrow, ribbon-like band having a first side and a second side, wherein the band is wound in a spiral to form a tube shaped member, with the first side of the band defining a lumen for the catheter; and
a flexible coating positioned on the second side of the band to establish a fluid-tight condition for the lumen of the catheter during articulation of the segment.

11. A segment as recited in claim 10 wherein the band has a substantially rectangular-shaped cross-section bounded by the first side, the second side and opposed first and second edges.

12. A segment as recited in claim 11 wherein a gap is formed between the first and second edges of the band when the band is wound into the spiral for the tube shaped member.

13. A segment as recited in claim 12 wherein the tube shaped member defines an axis and the gap defines a pitch angle (α) between the gap and the axis with the pitch angle (α) being in a range between forty-five and ninety degrees.

14. A segment as recited in claim 13 wherein the gap has a depth between the first side and the second side and a width substantially perpendicular to the depth with the depth being in a range of 0.1 mm to 0.2 mm and the width being in a range of 10 microns and 100 microns.

15. A segment as recited in claim 10 wherein the tube shaped member is made of a material selected from a group consisting of stainless steel and Nitinol.

16. A segment as recited in claim 10 wherein the flexible coating is made of a material from a group consisting of a polymer and nylon.

17. A method for manufacturing an articulating segment for a catheter which comprises the steps of:
providing a tube shaped member having an outer surface and an inner surface, wherein the inner surface defines a lumen for the catheter and the tube shaped member defines an axis;
cutting a helical shaped gap into the tube shaped member with the gap extending between the outer surface and the inner surface, wherein the gap defines a pitch angle (α) relative to the axis to establish a predetermined flexibility for the segment; and
positioning a flexible coating on the outer surface of the tube shaped member to cover the helical shaped gap and establish a fluid-tight condition for the lumen during articulation of the segment.

18. A method as recited in claim 17 wherein the gap defines a pitch angle (α) between the gap and the axis with the pitch angle (α) being in a range between forty-five and ninety degrees and further wherein the gap has a depth between the outer surface and the inner surface and a width substantially perpendicular to the depth with the depth being in a range of 0.1 mm to 0.2 mm and the width being in a range of 10 microns and 100 microns.

19. A method as recited in claim 17 wherein the cutting step is accomplished using a laser beam.

20. A method as recited in claim 17 wherein the predetermined flexibility allows the axis to bend with a radius of curvature of approximately fifteen mm.
